(19) United States
(12) Patent Application Publication
(10) Pub. No.: US 2008/0275403 A1
(43) Pub. Date: Nov. 6, 2008

(54) Dual syringe assembly

(76) Inventors: Armand Maaskamp, Coto de Caza, CA (US); Asim Syed, Irvine, CA (US); Ryan Maaskamp, Scottsdale, AZ (US)

Correspondence Address: Irell & Manella LLP, Ste. 900, 1800 Avenue of the Stars, Los Angeles, CA, 90067-4276, US

(21) Appl. No.: 12/082,291

(22) Filed: Apr. 9, 2008

Related U.S. Application Data
(60) Provisional application No. 60/927,761, filed on May 4, 2007.

(51) Int. Cl. A61M 5/19

(52) U.S. Cl. 604/191

(57) ABSTRACT

A dual syringe assembly that allows for two liquids to be sequentially injected through the same needle. By way of example, one syringe may contain a steroid and the other syringe may hold an anesthetic. The syringes are coupled to a needle by a connector. The needle can be inserted into a hand by medical personnel and manipulated until placed in contact with a tendon sheath. The steroid can be administered by depressing a plunger of the syringe that contains the steroid. The anesthetic can then be injected into the hand by depressing the plunger of the other syringe. The needle is then pulled out of the hand. The injection of the anesthetic flushes steroid from the needle and the Y-connector so that steroid is not ejected onto surrounding tissue when the needle is removed from the hand.

### CROSS-REFERENCE TO RELATED APPLICATION

The present application claims priority to Application No. 60/927,761 filed on May 4, 2007.

### BACKGROUND OF THE INVENTION

1. Field of the Invention

The present application relates to a valve connector for a dual syringe assembly.

2. Prior Art

Patients with rheumatoid arthritis are sometimes treated with an injection of a steroid directly into the tendon sheath of the patient's hand. When correctly administered the steroid can reduce inflammation and improve mobility of the hand. It has been found that steroids can cause atrophy of tissue surrounding the tendon sheath. When extracting the syringe needle some of the steroid may leak into the surrounding tissue and cause patient discomfort. One technique employed to reduce steroid leakage is to unscrew the syringe from the needle while the needle is still in the hand, attach a syringe full of anesthetic, and inject some anesthetic into the tendon. This is done to clear steroid out the needle before the syringe needle is pulled out of the hand. Having to unscrew one syringe and attaching another syringe to the inserted needle is a cumbersome technique for injecting anesthetic.

### BRIEF SUMMARY OF THE INVENTION

A connector valve assembly for a plurality of syringes. The connector includes a housing that has a pair of proximal ports, and a distal port in fluid communication with the proximal ports through first and second proximal channels. The assembly further has a first one-way valve located within the first proximal channel and a second one-way valve located within the second proximal channel.

### BRIEF DESCRIPTION OF THE DRAWINGS

FIG. 1 is an illustration of a syringe assembly;

FIG. 2 is an enlarged partially exploded view of the syringe assembly.

### DETAILED DESCRIPTION

Disclosed is a dual syringe assembly that allows for two liquids to be sequentially injected through the same needle. By way of example, one syringe may contain a steroid and the other syringe may hold an anesthetic. The syringes are coupled to a needle by a connector. The needle can be inserted into a hand by medical personnel and manipulated until placed in contact with a tendon sheath. The steroid can be administered by depressing a plunger of the syringe that contains the steroid. The anesthetic can then be injected into the hand by depressing the plunger of the other syringe. The needle is then pulled out of the hand. The injection of the anesthetic flushes steroid from the needle and the connector so that steroid is not ejected onto surrounding tissue when the needle is removed from the hand. Anesthetic can also be injected into the hand before injection of the steroid.

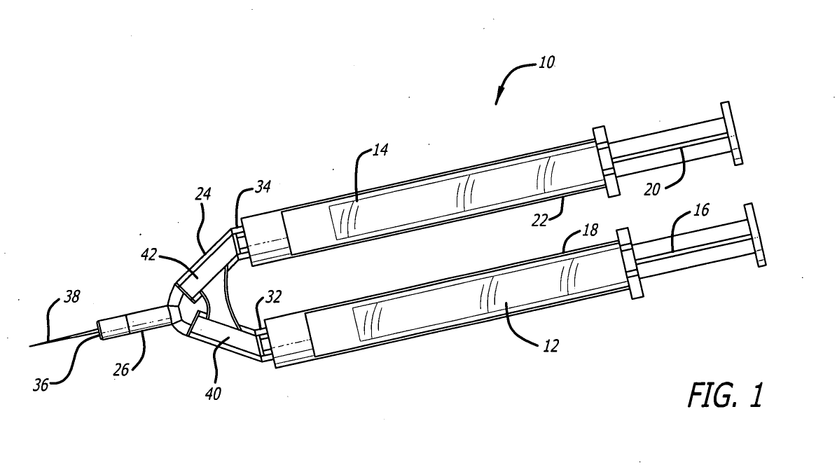

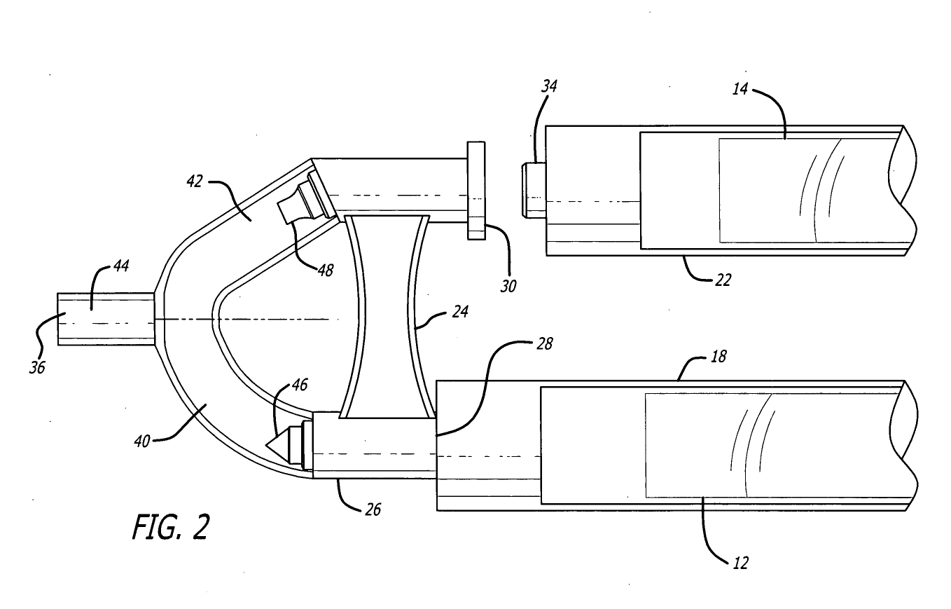

Referring to the drawings more particularly by numbers, FIGS. 1 and 2 show an embodiment of a dual syringe assembly 10. The assembly 10 includes a first syringe 12 and a second syringe 14. The first syringe 12 may have a plunger 16 that can move inside a tube 18. Likewise, the second syringe 14 may have a plunger 20 that is moved inside a tube 22. The tubes 18 and 22 can be constructed from transparent or semi-transparent plastic material and have markings (not shown) that provide an indication of the volume of fluid within the syringe. The plungers 16 and 20 can also be constructed from a plastic material.

The assembly 10 may include a connector valve assembly 24 attached to the syringes 12 and 14. The connector 24 includes a housing 26 that has a first proximal port 28 and a second proximal port 30. The first proximal port 28 is connected to a flange 32 of the first syringe 12. The second proximal port 30 is connected to a flange 34 of the second syringe 14. The housing 26 and flanges 32 and 34 are constructed so that a user can easily connected and disconnect the syringes 12 and 14 from the connector assembly 24.

The connector assembly housing 26 has a distal port 36 that is in fluid communication with a needle 38 that is attached to the housing 26. The distal port 36 and needle 38 are in fluid communication with the syringes 12 and 14 through a pair of proximal channels 40 and 42 and a distal channel 44. Each channel 40 and 42 may have a check valve 46 and 48, respectively. The check valves 46 and 48 prevent fluid from one syringe 12 or 14 from being injected into the other syringe 14 or 12. By way of example, the check valves 46 and 48 may be duck-bill valves constructed from a rubber or plastic material. The connector 24 may be constructed from a molded plastic material.

The syringes 12 and 14 can be arranged in a parallel relative relationship as shown, or can be attached to the connector 24 so as to be at an oblique angle relative to each other. One of the syringes 12 or 14 can contain a steroid and the other syringe 14 or 12 may contain an anesthetic. The assembly 10 may be used to sequentially inject the steroid and anesthetic into a hand.

In operation, a user can attach the needle 38 and syringes 12 and 14 to the connector assembly 24. The needle 38 can be attached to the connector assembly 24 before or after attachment of the syringes 12 and 14 to the assembly 24. The needle 38 can then be inserted into a patient. The plunger 16 of the first syringe 12 is depressed to inject a first medical fluid into the patient through the needle 38. The first medical fluid flows through the first one-way valve 46 but is blocked from flowing into the second syringe 14 by the second one-way valve 48. By way of example, the first medical fluid may be a steroid. The plunger 20 of the second syringe 14 is then depressed to inject a second medical fluid into the patient. The second medical fluid flows through the second one-way valve 48 but is blocked from flowing into the first syringe 14 by the first one-way valve 46. By way of example, the second medical fluid may be an anesthetic. The needle 38 is then pulled out of the patient's hand.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art.

What is claimed is:

1. A connector valve assembly for a plurality of syringes, comprising:
a housing that includes a pair of proximal ports, and a distal port in fluid communication with said proximal ports through first and second proximal channels;
a first one-way valve located within said first proximal channel; and,
a second one-way valve located within said second proximal channel.

2. The assembly of claim 1, wherein said first and second one-way valves are duck bill valves.

3. The assembly of claim 1, wherein said housing has a Y-shape.

4. The assembly of claim 1, further comprising a needle that is attached to said housing and is in fluid communication with said distal port.

5. The assembly of claim 1, wherein said housing includes a pair of proximal ports that are attached to the syringes.

6. A syringe assembly, comprising:
a first syringe;
a second syringe;
a connector housing that is attached to said first and second syringes, said connector housing includes a first proximal port coupled to said first syringe, a second proximal port coupled to said second syringe, and a distal port in fluid communication with said first and second proximal ports through first and second proximal channels;
a first one-way valve located within said first proximal channel; and,
a second one-way valve located within said second proximal channel.

7. The assembly of claim 6, wherein said first and second one-way valves are duck bill valves.

8. The assembly of claim 6, wherein said connector housing has a Y-shape.

9. The assembly of claim 6, further comprising a needle that is attached to said connector housing and is in fluid communication with said distal port.

10. A method for injecting a first medical fluid and a second medical fluid into a patient, comprising:
attaching a connector valve assembly to a first syringe that contains a first medical fluid and a second syringe that contains a second medical fluid, the connector valve assembly including a first one-way valve and a second-one way valve, a needle extends from the connector valve assembly;
inserting the needle into a patient;
pressing the first syringe to inject the first medical fluid into the patient, the first medical fluid flowing through the first one-way valve; and, pressing the second syringe to inject the second medical fluid into the patient, the second medical fluid flowing through the second one-way valve.

11. The method of claim 10, wherein the first medical fluid is a steroid and the second medical fluid is a anesthetic.

12. The method of claim 10, wherein the second medical fluid is injected into the patient while the needle is pulled out of the patient.

13. The method of claim 10, further comprising injecting the second medical fluid into the patient before injection of the first medical fluid.

14. The method of claim 10, wherein the needle is attached to the connector valve assembly after attachment of the first and second syringes to the connector valve assembly.
